(19) United States
(12) Patent Application Publication
(10) Pub. No.: US 2024/0416057 A1
(43) Pub. Date: Dec. 19, 2024

(54) SYSTEM AND METHODS FOR DYNAMICALLY CONTROLLING OPERATION OF A MECHANICAL VENTILATOR FOR AUTOMATIC INTERVENTION DURING A DETECTED RESPIRATORY EPISODE

(71) Applicant: Breas Medical AB, Molnlycke (SE)

(72) Inventors: William A. Truschel, Oakmont, PA (US); Harout Rafi Stepanian, Molnlycke (SE)

(73) Assignee: Breas Medical AB, Molnlycke (SE)

(21) Appl. No.: 18/741,510

(22) Filed: Jun. 12, 2024

Related U.S. Application Data
(60) Provisional application No. 63/521,574, filed on Jun. 16, 2023.

(51) Int. Cl. A61M 16/00

(52) U.S. Cl. CPC A61M 16/024; A61M 16/0003; A61M 2202/0007; A61M 2202/0208; A61M 2205/3303; A61M 2205/3327; A61M 2205/3553; A61M 2205/502; A61M 2205/583; A61M 2230/005; A61M 2230/04; A61M 2230/20; A61M 2230/42; A61M 2230/43; A61M 2230/46

(57) ABSTRACT

A system and method are provided for dynamically controlling the operation of a mechanical ventilator. A saved prescription includes a base or therapeutic ventilation configuration, intervention logic for identifying an intervention entrance condition, and an intervention configuration. The ventilator system initially operates in the base configuration, continuously monitors readings from patient biometric sensors, continuously determines, according to the intervention logic and the monitored readings, if an intervention entrance condition is present. If the entrance condition is present, the system dynamically reconfigures into the intervention configuration. Intervention exit conditions are also programmed wherein during operation in the intervention configuration, the method continuously monitors readings from the biometric sensors, determines, according to the intervention logic and the monitored readings, if an intervention exit condition is present, and if exit conditions are present, reconfigures the ventilator system into the base configuration.

### CROSS-REFERENCE TO RELATED APPLICATIONS

This application claims the benefit of U.S. Provisional Patent Application Ser. No. 63/521,574 filed Jun. 16, 2023, the entire contents of which are incorporated herein by reference.

### BACKGROUND OF THE DISCLOSURE

(1) Field of the Invention: The instant invention relates to mechanical ventilator systems, and more particularly to dynamic control of a mechanical ventilator which is not based on algorithmic analysis.

(2) Description of Related Art: The state of the art in the practice of mechanical ventilation is performed under close supervision of a medical care professional in the intensive care unit (ICU). In the ICU, the patient is closely and continuously monitored with external biometric sensors, and the ventilator's alarm system. Any alert provokes an immediate response by the medical professional nearby and that caregiver can assess the patient, make any necessary changes to the therapy or therapy settings and watch, and wait for a positive response and healthy outcome for the patient before a medical emergency develops.

In contrast, when a patient is diagnosed with chronic respiratory insufficiency or chronic respiratory failure, the patient is often sent home with a mechanical ventilator on a fixed prescription. Although biometric sensors and the ventilator alarm system is still active in the home, it is common that no medical professional is nearby to change therapy or change the therapy settings to invoke or initiate a more effective care regimen for the patient. Follow up appointments are typically 6 to 8 weeks after discharge from the care of the intensivists in a hospital or clinic and regularly every 6 to 12 months after the first follow up.

Recent developments in connected care, allow a caregiver to review a patient remotely with a digital dashboard, but this review is often sporadic and spaced days, weeks, or months apart. When we consider that the solution involves allowing the clinician to change the settings for a critical patient, we incur a higher risk, and these remote unsupervised changes are discouraged. In fact, the message from the global regulatory bodies to allow a change to therapy for critical patients when the caregiver is remote is, “don't do it.”

Furthermore, the remote settings solution is not scalable as it requires the constant attention of expert manpower to monitor a large population of homecare patients within a health care system. Due to these challenges, it is expected that patients who need critical attention to a respiratory abnormality will not thrive under the current state of the art.

Due to the nature of the respiratory disease of the patient that may be considered progressive or in the category of a relapsing/remitting type from critical to stable at unknown frequency, it is often likely that therapy setting changes will lag behind the needs of the patient when the intervals of remote monitoring and patient follow-ups are considered.

Some patients with chronic respiratory diseases may fall into a category of having predictable relapses in disease severity such as when a pathogen catalyzes inflammation within a patient who already has obstructive lung disease or when a patient experiences metabolic overload due to exercise and has an episode of dyspnea. Today these episodes are addressed with caregiver interventions such as changing to a back up prescription manually, or if able, by the patient intervening by calling for help to a caregiver who may administer an alternative treatment setting on the ventilator or assist via aerosolized treatment in conjunction with ventilator. However, these manual interventions are not always possible or cannot be given in real time and thus do not represent favorable outcomes for the patient.

### SUMMARY OF THE DISCLOSURE

If the caregiver is not present or if the episode disables the patient severally or if the patient is generally incapable of intervening due to a physical impairment, we suggest that these interventions can be programmed into the mechanical ventilator in a novel way described here.

The present disclosure describes a programmable intervention system where the clinician may prescribe and individualize both the detection of an episodic need and the machine's response to that episode. The prescribing clinician may define a logical combination of any biosensor measurements crossing a threshold and/or any measured device parameters crossing a threshold to describe an “entrance” condition for an intervention state and similarly the clinician may further define a different logical combination for the “exit” condition to return to the base or therapeutic state.

Once entering the intervention state, the clinician can program the device to deliver a modified prescription suitable for addressing the needs of the patient during the episode of distress. The interventional prescription can simply be a higher or lower value of one particular setting within the same therapeutic mode, or a completely new set of settings in a completely new mode of ventilation. For example, in the base therapy state, the patient may be receiving Continuous Positive Airway Pressure (CPAP) with particular operational parameters, but in the intervention state the machine may automatically switch to a Volume Assisted Control (AC) mode of ventilation.

The present invention intends to overcome the issues of inappropriate response time incurred due to remote monitoring and more specifically the issue of “no response” when caregivers are absent, or the patient is unable to intervene on his own. The clinician ultimately knowing what is best for an individual or class of patients and has full authority over the intervention actions. There are no hidden algorithms contained within the device to automatically make a clinical assessment of the patient and the device does not decide what an appropriate response could be. The device will simply only operate according to the direction of the programming clinician and there is full transparency of how, when, and why a setting will change. The control system adjusting the parameter or parameters is constrained to approved ranges ascribed for the device and the class of patient. In other words, the clinician cannot intervene with a prescription that is not already allowed under the device's intended use or qualified range of settings. The procedures and techniques including the stability analysis, verification, and validation of the basic device will ensure compliance with the applicable safety standards and that all associated safety risks have been managed as far as possible in the design.

According to exemplary embodiments of the invention, a system and method for dynamically controlling the operation of a ventilator system includes accessing a saved prescription for automatic control of the ventilator system wherein the prescription includes a base ventilation configuration, intervention logic for identifying an intervention entrance condition of the target person, and an intervention configuration. The method further includes configuring and operating the ventilator system in the base configuration, continuously monitoring readings from a plurality of biometric sensors and determining, according to the intervention logic and the monitored readings, if an intervention entrance condition is present, and if an intervention entrance condition is present, reconfiguring the ventilator system according to the intervention configuration. The intervention logic may further include parameters for identifying an intervention exit condition wherein during operation of the ventilator system in the intervention configuration, the method further includes the steps of continuously monitoring readings from the plurality of biometric sensors, determining, according to the intervention exit logic and based on the monitored readings, if an intervention exit condition is present, and if an intervention exit condition is present, reconfiguring the ventilator system to provide the base mechanical ventilation to the target person according to the base configuration.

In some embodiments, the base configuration and the intervention configuration may each comprise a prescription set that includes an operational mode and the associated values of operational parameters associated with that particular operational mode. The operational mode of the base configuration or intervention configuration may include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational parameters associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP). In many instances the base mode and intervention mode may be the same, but the intervention mode may include one of more differences in operation parameters that are all pre-determined by the prescribing clinician to affect positive outcomes when the patient is in need.

In some embodiments, the biometric sensor readings may include, but are not limited to the following: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 illustrates a ventilator system;

FIG. 2 illustrates lungs and the inspiration and expiration flows in and out of the lungs;

FIG. 3 illustrates a schematic of a programmable interventional ventilation system in accordance with the teaching of the present invention;

FIG. 4 illustrates setting of a prescription parameter as a fixed value in a non-interventional state;

FIG. 5 illustrates interventional prescription operation of the system in a common mode between therapeutic and base modes and with a single interventional parameter showing activation of an interventional prescription setting, and the difference between the base/therapeutic setting and the interventional setting in the range of allowable settings, and a prompt for setting entry and exit conditions for the dynamic intervention;

FIG. 6 illustrates setting of the entrance and exit condition intervention logic with parameter selection, and entry and exit thresholds;

FIG. 7 illustrates a flow diagram for operation of the system in dynamic intervention mode;

FIG. 8 illustrates operation of the system in a second multi-mode and parameter embodiment, and manual setting of multiple different prescription operational modes in a non-interventional state;

FIG. 9 illustrates interventional operation of the system where operating modes and/or multiple parameters are selected and programmed, and activation of an interventional prescription mode;

FIG. 10 illustrates setting of the entrance and exit condition intervention logic with parameter selection, and entry and exit thresholds;

FIG. 11 illustrates a flow diagram for operation of the system in dynamic intervention mode according to the second embodiment;

FIG. 12 is a chart of common ventilator operating modes and associated parameters set for each mode along with common values and units.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

Some definitions and abbreviations that will be helpful for this application including the following:

Inspiratory flow refers to the flow of air entering into and flowing towards the lungs. Expiratory flow refers to the flow of air exiting the lungs and flowing towards the glottis. Rise Time is the rate at which the pressure ramps up to the prescribed or determined pressure level. The pressure generally rises during inspiration. The rise time of pressure can affect the flow rate and in particular the inspiratory flow rate and in more particular the peak inspiratory flow.

Fall Time is the rate at which the pressure ramps down to a determined pressure level. The pressure generally decreases during expiration. The fall time of pressure can affect the flow rate and in particular the expiratory flow rate and in more particular the peak expiratory flow.

Prescribed Pressure or Pressure Dosage is the amount of pressure that the ventilator ramps up to during use of the ventilator and is usually prescribed by a medical provider. Units of pressure are usually in the form of cm H₂O or centimeters of water column. Prescribed pressures generally range from 5-25 cm H₂O, and generally do not exceed 30 cm H₂O.

Mechanical ventilators are operational in several different modes. The most common modes of mechanical ventilation include but are not limited to: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

Volume control modes are generally favored for greater ventilation control, while pressure control modes are favored for assisted or spontaneously breathing patients. Both types of modes have advantages and disadvantages that are mainly related to the flow and pressure patterns of gas delivery.

Mechanical ventilators have many parameters that clinicians can adjust to treat a patient's condition, including but not limited to:

| Parameter | Value | Units |
|---|---|---|
| Peak Inspiratory Pressure | 0-99 | cm H2O, mbar, hPa |
| Peak Inspiratory Flow | 0-200 | L/min |
| Inhaled Tidal Volume | 0-2000 | ml |
| Exhaled Tidal Volume | 0-2000 | ml |
| Respiratory Rate | 0-120 | BPM |
| Minute Volume | 0-40 | L/min |
| % Spontaneous Trigger | 0-100 | % |
| Asynchrony Index | 0-100 | % |
| Lung Compliance | 1-200 | ml/cm H2O, ml/mbar, |
| | | ml/hPa |
| Static Lung Compliance** | 1-200 | ml/cm H2O, ml/mbar, |
| | | ml/hPa |
| Static Lung Resistance** | 1-200 | cmH2O/Lps, mbar/Lps, |
| | | hPa, Lps |
| Plateau Pressure** | 0-99 | cm H2O, mbar, hPa |
| Alveolar Pressure | 0-99 | cm H2O, mbar, hPa |
| Expiratory Resistance | 1-200 | cmH2O/Lps, mbar/Lps, |
| | | hPa, Lps |
| Inspiratory Resistance | 1-200 | cmH2O/Lps, mbar/Lps, |
| | | hPa, Lps |
| iPEEP | 0-40 | cm H2O, mbar, hPa |
| FiO2 | 21-100 | % |
| SpO2 | 5-100 | % |
| Heart Rate | 18-321 | BPM |
| ETCO2 | 5-150 | mmHg |
| TcCO2 | 5-150 | mmHg |
| Peak Expiratory Flow | 0-200 | L/min |
| I:E Ratio | 2:1:1:99 | |
| Expiratory Time | 1-60 | sec |
| RSBI | 8-999 | Breaths/min/L |
| Vt/kg IBW | 1-30 | cc/KgIBW |
| Flow Bias Ratio* | 10:1:1:10 | Insp:Exp |
| Average Leak | 0-200 | L/min |
| Leak Ratio to Normal | 0.0:10 | None |
| AHI (Apnea Hypopnea Idx) | 0-300 | per hour |
| Expiratory Time Constant | 0.1-10 | sec |
| Resp Rate-Oxygenation (ROX) | 0-99 | points |

Abbreviations associated with the above are set forth below.

| Parameter Name | Abbreviation |
|---|---|
| Peak Inspiratory Pressure | PIP |
| Peak Inspiratory Flow | PIF |
| Tidal Volume | Vt |
| Respiratory Rate | RR |
| Minute Volume | MVexp |
| % Spontaneous Trigger | % Spon Trigger |
| Asynchrony Index | Asynch Idx |
| Lung Compliance | Clung |
| Static Lung Compliance** | Cstat |
| Static Lung Resistance** | Rstat |
| Plateau Pressure** | Pplat |
| Alveolar Pressure | Palv |
| Expiratory Resistance | Rexp |
| Inspiratory Resistance | Rinsp |
| Intrinsic PEEP | iPEEP |
| Fraction of Inhaled O2 | FiO2 |
| Saturation of Peripheral O2 | SpO2 |
| Heart Rate | HR |
| End Tidal Carbon Dioxide | ETCO2 |
| Transcutaneous Carbon Dioxide | TcCO2 |
| Peak Expiratory Flow | PEF |
| Inspiratory Expiration Ratio | I:E Ratio |
| Rapid Shallow Breathing Index | RSBI |
| Tidal Volume per Ideal Body Weight | Vt/kg IBW |
| Flow Bias Ratio* | PEF/PIF Ratio |
| Average Leak | Ave Leak |
| Leak Ratio to Normal | Leak Ratio |
| Apnea Hypopnea Index | AHI |
| Expiratory Time Constant | TCexp |
| Inhaled Tidal Volume | Vti |
| Exhaled Tidal Volume | Vte |
| Resp Rate-Oxygenation (ROX) | ROX |

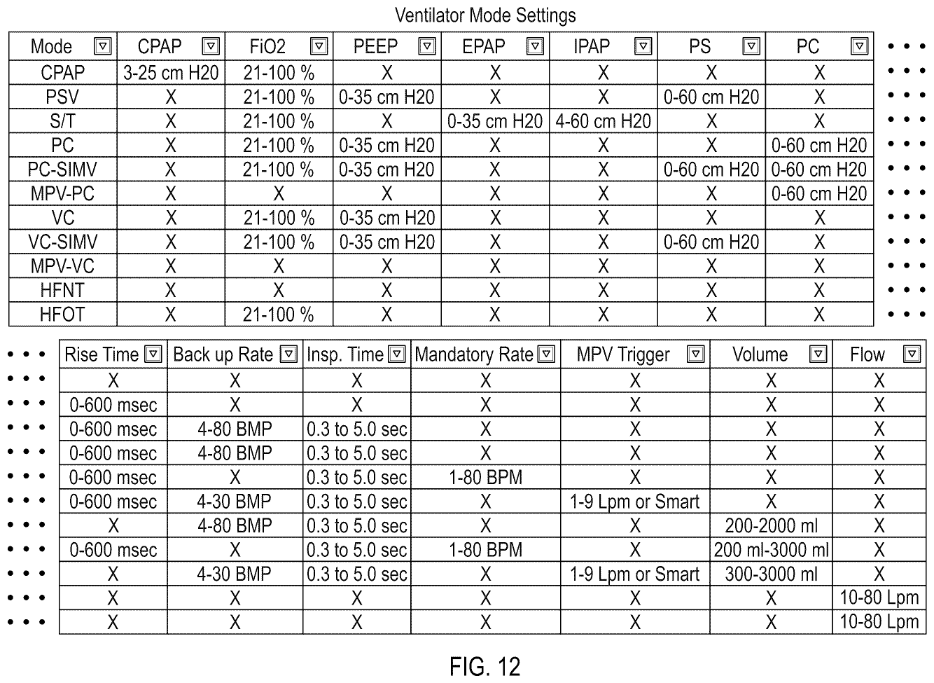

Finally, parameters to be set depend on modes. An exemplary setting chart is set forth in FIG. 12, where the settings names are the column headers.

Additional definitions are also set forth below.
Respiratory rate (RR): The number of breaths a patient takes per minute
Tidal volume (VT): The normal range is 6-8 mL/kg
Positive end-expiratory pressure (PEEP)
Inspiratory airflow (V′)
Percentage of inspired oxygen (FiO2): Normal is 35-50%, but can be increased to 75-100% if needed
Air pressure (SetP)
Peak inspiratory pressure (PIP): The sum of PEEP and SetP
Peak pressure
Inspiratory time: Inspiratory Time is the length of time during which the ventilator delivers the inspiratory pressure.
Inspiratory-to-expiratory ratio
Time of pause
Trigger sensitivity
Support pressure
Expiratory trigger sensitivity

Ventilators can also produce ventilator-derived parameters, which can be used to guide ventilatory strategies and detect problems with the ventilator or changes in the patient:
Intrinsic PEEP (PEEPi): The residual pressure when the expiratory phase isn't fully completed
Plateau pressure (Pplat): Equal to alveolar pressure when airflow is zero
Transpulmonary pressure
Transpulmonary driving pressure (ΔP)
Mechanical energy
Mechanical power and intensity
Pressure-time product per minute (PTP)

Patients on mechanical ventilation are usually monitored in an intensive care unit (ICU) and have monitors that measure several values related to respiration, including but not limited to: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

As will be described in more detail hereinbelow, the invention provides a novel, programmable intervention system and methodology where the clinician may prescribe and individualize both the detection of a respiratory episode and the ventilator machine's response to that episode. The prescribing clinician may define a logical combination of any biosensor measurements crossing a threshold and/or any measured device parameters crossing a threshold to describe an “entrance” condition for an intervention state and similarly the clinician may further define a different logical combination for the “exit” condition to return to the base or therapeutic state.

Once entering the intervention state, the clinician can program the device to deliver a modified prescription suitable for addressing the needs of the patient during the episode of distress.

In a first embodiment, the system is programmed with a base “therapeutic” mode having a set operational mode and/or parameters, and an interventional prescription can simply be a higher or lower value of one particular setting within the same therapeutic mode, or a completely new set of settings in a completely new mode of ventilation. For example, in the base therapy state, the patient may be receiving Continuous Positive Airway Pressure (CPAP) with particular operational parameters, but in an intervention state the machine may automatically switch to a Volume Assisted Control (VAC) mode of ventilation with different operational parameters.

The present invention overcomes the issues of inappropriate response time incurred due to remote monitoring and more specifically the issue of “no response” when caregivers are absent, or the patient is unable to intervene on his own.

There are no underlying algorithms contained within the device programming to make clinical assessments of the patient, and the device does not decide what an appropriate response could be. Biometric and operating conditions are measured, threshold values are set, and a prescribed response is set. The system will simply only operate according to the direction of the programming clinician. There is full transparency of how, when, and why a setting will change.

An important distinction with Programmable Ventilation according to the present disclosure, is that modification or adjustment of the ventilator's mode and/or operational parameters is solely controlled by the heath data signals monitored and displayed by the device without providing any “diagnostic function” for analysis of the patient's biological condition and physiological state.

For example, an increase in applied pressure is determined necessary when the patient's CO₂ exceeds a particular threshold. In this example, the device makes no diagnostic determination that the patient is hypo-ventilating or has undergone respiratory failure, the control is simply based on the sensor's measurement. The device will not manage the patient according to any incorporated diagnostic algorithm, rather it will change operation modes, adjust one or more prescription parameters, or both, to intervene in a manner previously prescribed by the clinician.

The control system adjusting the parameter or parameters is constrained to approved ranges ascribed and programmed for the device and the class of patient. In other words, the clinician cannot intervene with a prescription that is not already allowed under device's intended use or qualified range of settings (saved prescription). The procedures and techniques including the stability analysis, verification, validation will ensure compliance with the applicable safety standards and that all associated safety risks have been managed as far as possible in the design.

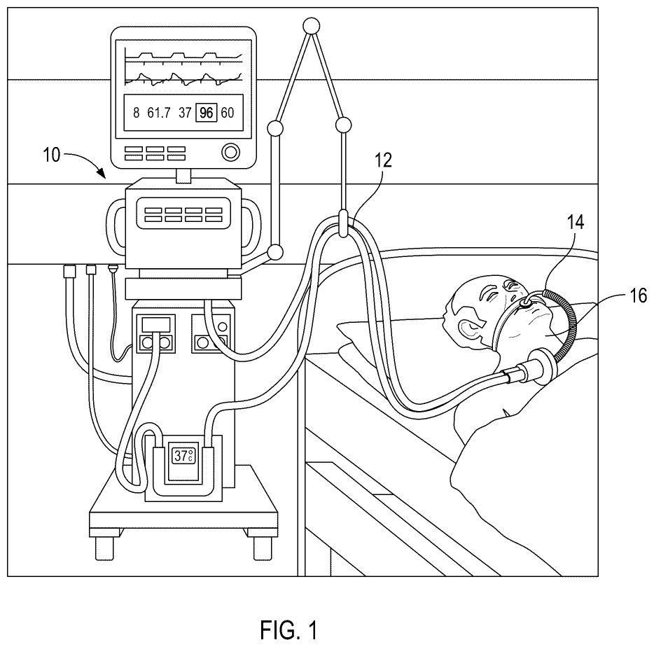

Turning now to the drawing figures, FIG. 1 illustrates a basic ventilator system 10 that provides pressurized air through the tube 12 into an airway adaptor 14, such as a tube or mask, to the user/patient 16. In some instances, a mask is not used, where the tube is directly fed into the trachea, such as a tracheostomy.

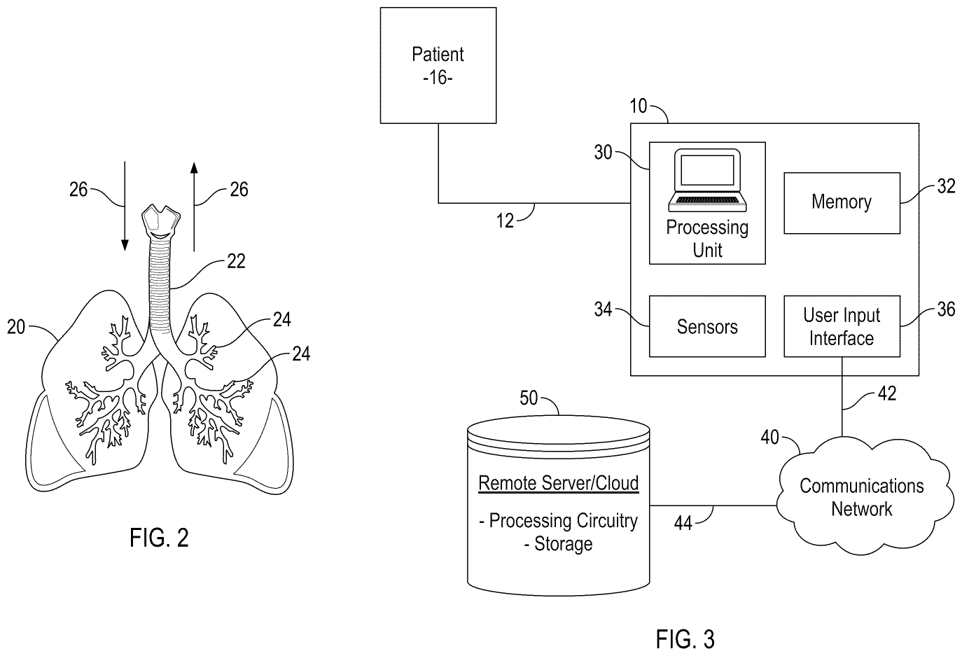

FIG. 2 illustrates lungs 20, including the trachea 22 and the bronchi of the lungs 24. The inspiration flow path 26 travels into the trachea 22 and into bronchi 24, whereas the expiration flow path 28 travels or flows out from the lungs 20 and bronchi 24 into and out of the trachea 22.

FIG. 3 illustrates a schematic of an interventional mechanical ventilation system 100 that includes a ventilator system 10, which includes a processing unit 30 configured to receive input operating parameters (as set forth hereinabove), via a user/patient input interface 36, implement intervention logic tuples and protocols, as well as direct and analyze sensor data captured by sensors 34, recall and place data into memory 32, and direct communications over a network 40 to remote server/cloud 50 that also includes processing circuitry and storage. Directed communications 42 can be made to and from the communications network, which can make directed communications 44 to and from the remote server/cloud 50. Cloud computing is generally understood in the art to mean the delivery of computing services, including servers, storage, databases, networking, software, analytics, and intelligence over the Internet (“the cloud”) to offer faster innovation, flexible resources, and economics of scale.

According to exemplary embodiments of the invention, a system and method for dynamically controlling the operation of a ventilator system includes accessing a saved prescription for automatic control of the ventilator system wherein the prescription includes a base ventilation configuration, intervention logic for identifying an intervention entrance condition of the target person, and an intervention configuration. The method further includes configuring and operating the ventilator system in the base configuration, continuously monitoring readings from a plurality of biometric sensors and determining, according to the intervention logic and the monitored readings, if an intervention entrance condition is present, and if an intervention entrance condition is present, reconfiguring the ventilator system in the intervention configuration. The intervention logic may further include parameters for identifying an intervention exit condition wherein during operation of the ventilator system in the intervention configuration, the method further includes the steps of continuously monitoring readings from the plurality of biometric sensors, determining, according to the intervention logic and based on the monitored readings, if an intervention exit condition is present, and if an intervention exit condition is present, reconfiguring the ventilator system to provide the base mechanical ventilation to the target person according to the base configuration. Furthermore, the exit condition is not the machine's clinical assessment that the intervention is no longer necessary, but that combination of respiratory measurements which the clinician has deemed the exit condition describing when the base or therapeutic therapy is sufficient to treat the patient until the entrance condition is met again.

In some embodiments, the base configuration and the intervention configuration may each comprise one or more predetermined operational parameters and/or operating modes which include but are not limited to the following: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T).

The operational parameters associated with these modes include but are not limited to: Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations of the modes noted and the operational parameters set forth above.

In some embodiments, the biometric sensor readings may include, but are not limited to the following: heart rate, respiratory rate, blood pressure, Oxygen Saturation (SpO₂) and End-Tidal Carbon Dioxide (ETCO₂).

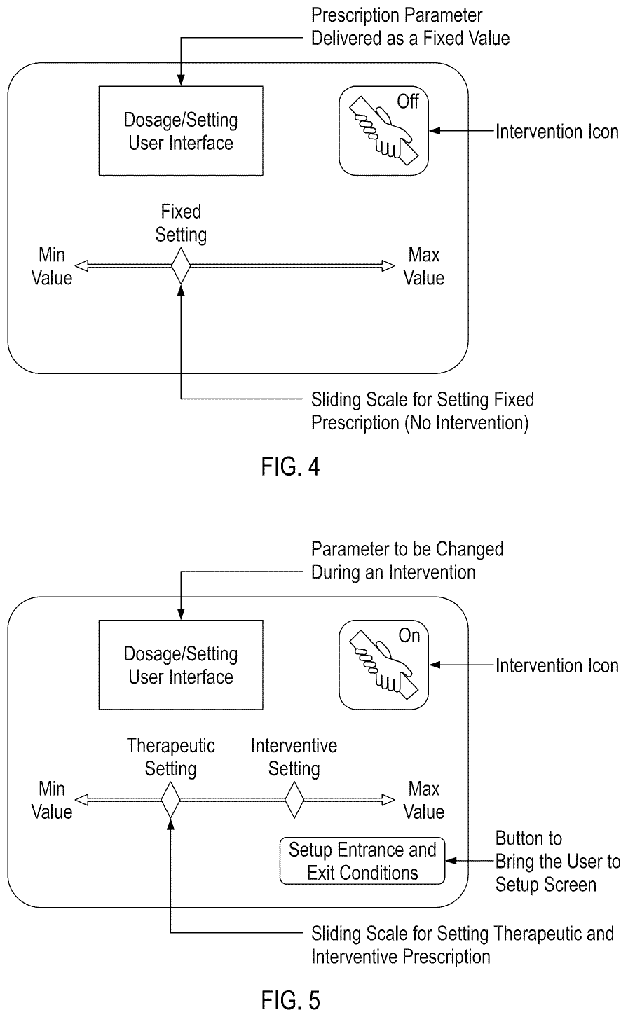

Referring now to FIGS. 4-7 a first exemplary configuration of the system and methodology are illustrated and described.

### Setting Up the Intervention Program

### Changing a Single Therapy Setting to Intervene

In the simplest form only a single setting (parameter) is changed during an intervention. This can be done while choosing the parameter setting in the machine's user interface.

First, we decide which setting will be involved in the intervention strategy. (For example, an increase to Pressure Support in Pressure Support Ventilation for hypoventilation (Minute Ventilation below a threshold or CO₂ above a threshold) in patient with neuromuscular disease, or an Increase in FiO2 for patients with hypoxemia (low SpO₂ and high heart rate), or an increase in EPAP for patients with obstructive sleep apnea (Apnea-Hypopnea index above a threshold), or an increase in Flow Trigger Sensitivity in patients with lung injury having episodes of increased respiratory drive (work of breathing above threshold.)

When intervention involves only a single setting, that setting is changed from a fixed value to a pair of values (See FIGS. 4 and 5). As a matter of example, the user interface for setting a parameter, may contain a switch to change the setting between a fixed setting (the same dosage is always delivered) or an interventional setting (see also FIGS. 4 and 5, Intervention Icon ON/OFF). When the intervention program is enabled, the user selects two values for the setting. A therapeutic setting and an intervention setting.

The goal of the intervention is to treat a patient episode with an intervention prescription when the episode is active and to treat a patient with a therapeutic prescription when an episode is not active or has subsided.

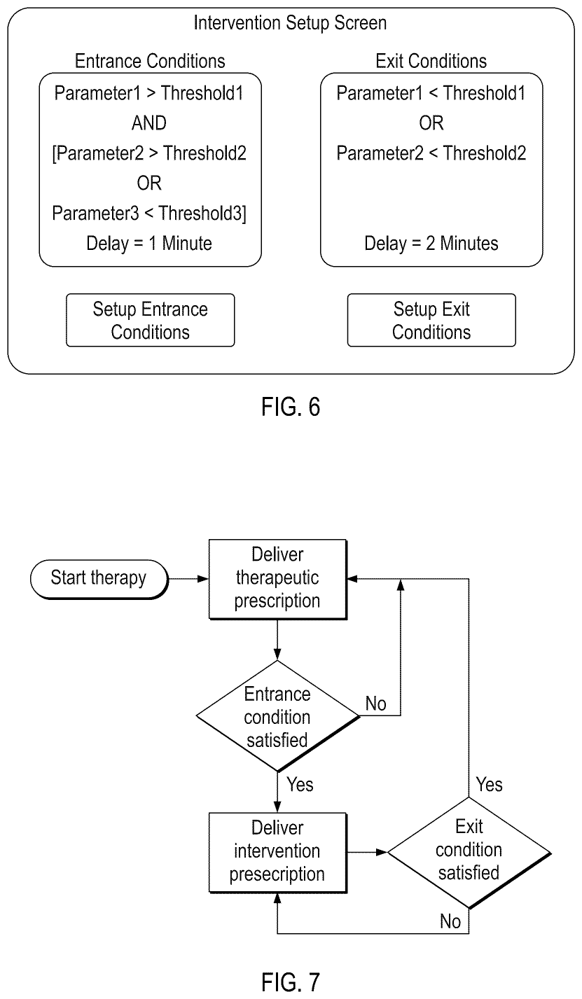

Turning to FIG. 6, the logic conditions for setting up entrance and exit conditions are selected from a menu type interface, where one or more conditions are setup by the user. The user must select:
1. Parameters involved in the detection of an episode.
2. Thresholds for each parameter to determine if an episode is active (enter intervention state) or if the episode has subsided (exit intervention state).
3. Comparison Type (less than, greater than, less than or equal to, greater than or equal to).
4. The delay value.

The entrance condition delay value determines the period in the therapeutic state through which the entrance condition must be true before the machine enters the intervention state.

The exit condition delay value determines the period in the intervention state through which the exit condition must be true before the machine reverts to the therapeutic state.

Once initiated the system operates according to the flow diagram as illustrated in FIG. 7 delivering either the base therapeutic ventilation or the interventional ventilation depending on the sensed biometrics, entry conditions and exit conditions.

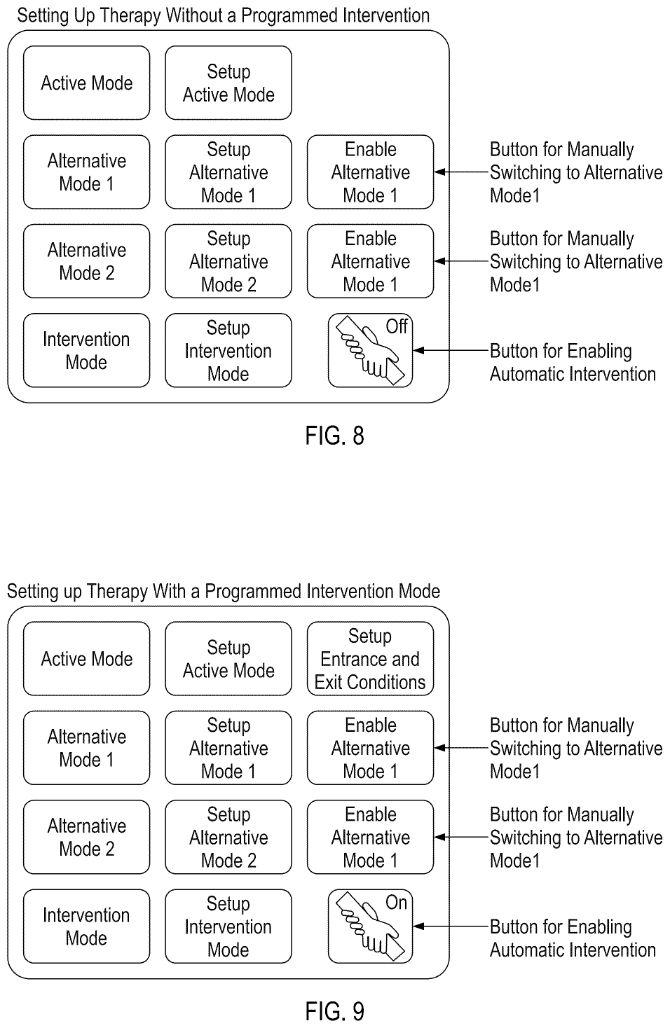

Referring now to FIGS. 8-11, a second exemplary configuration of the system and methodology are illustrated and described.

Employing the Intervention as a change to more than one value and/or a new mode and changed values.

If an intervention involves more than one setting, the system allows for both manual switching between modes of ventilation and automatic switching by means of selecting a programmed intervention.

The system memory 32 allows more than one set of prescriptions to be stored along with respective entrance and exit conditions. One set of settings shall be referred to as the active prescription mode. When therapy is started, the active prescription is delivered to the patient. The user (with permission or access to change the prescription) may manually change the prescription to an alternative mode by enabling a previously and professionally setup set of prescriptions (See FIGS. 8 and 9). These alternative prescriptions can be referred to as “Alternative Mode1” or “Alternative Mode2” or they can be given custom names such as “Daytime Prescription”, “Rescue Prescription”, etc. These alternative prescriptions can be enabled to configure the machine to deliver a different prescription to the patient when therapy is active.

Enabling an alternative prescription, makes the alternative prescription the active prescription and it makes the previously active prescription an alternative prescription. This feature of switching prescriptions manually is the state of the art and provides competent users the ability to manually intervene or modify the prescription in bulk with simple actions.

In the present embodiment, a programmed intervention is introduced that does not require an expert to manually detect the need to change the prescription in bulk.

When the intervention mode is enabled or “ON”, the setup involves three items.

A complete set of parameters and values along with the intervention mode. For example, the active mode can be CPAP with FiO2=21% and the Intervention mode can be Assist Volume Control with Volume set to 500 ml, Inspiratory Time set to 1 second, Back Up Rate set to 15 BPM, FiO2 set to 40%, etc.

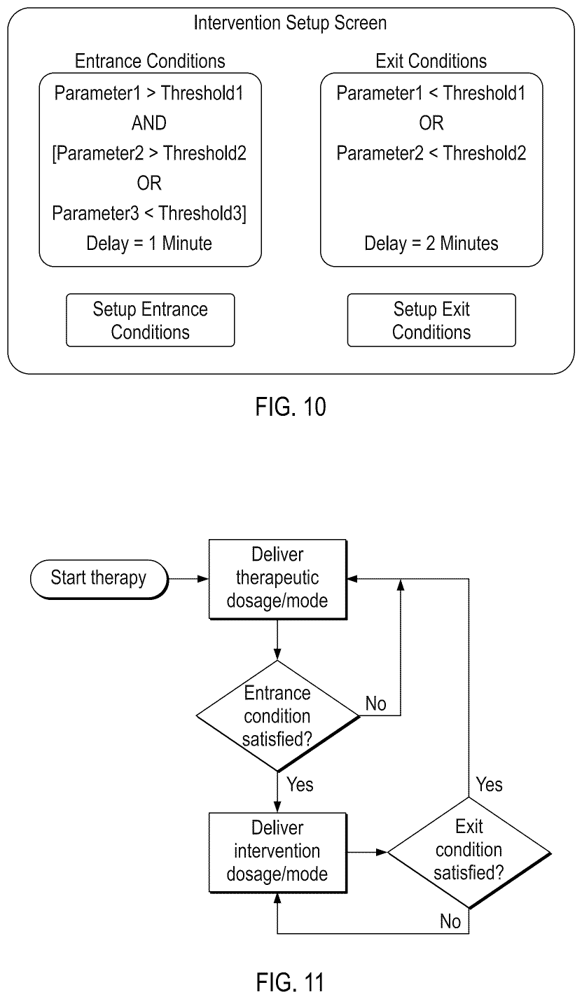

When the intervention mode is active, the user must set the entrance and exit conditions just as if the user was setting up a single parameter intervention (See FIG. 10).

Once initiated the system operates according to the flow diagram as illustrated in FIG. 11 delivering either the base therapeutic ventilation according to a first prescription mode and parameters, or one of the other programmed prescriptions (alternative modes and parameters) depending on the sensed biometrics, entry conditions and exit conditions.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method for dynamically controlling the operation of a ventilator system in providing mechanical ventilation to a target person, comprising:
accessing a prescription for automatic control of the ventilator system in providing mechanical ventilation to the target person, the prescription comprising:
a base configuration for configuring the ventilator system to provide a base mechanical ventilation to the target person,
intervention logic for identifying an intervention entrance condition of the target person, and
an intervention configuration for configuring the ventilator system to provide interventional mechanical ventilation to the target person;
configuring the ventilator system according to the base configuration;
operating the ventilator system according to the base configuration;
during operation of the ventilator system while configured according to the base configuration, continuously monitoring readings from a plurality of biometric sensors corresponding to the target person;
determining, according to the intervention logic and based on the monitored readings, if an intervention entrance condition is present in the target person; and
if an intervention entrance condition is present, dynamically reconfiguring the ventilator system according to the intervention configuration to provide interventional mechanical ventilation to the target person according to the intervention configuration.

2. The method of claim 1, wherein the intervention logic further comprises:
intervention logic for identifying an intervention exit condition of the target person; and
wherein the method further comprises, during operation of the ventilator system while configured according to the intervention configuration, continuously monitoring readings from a plurality of biometric sensors corresponding to the target person;
determining, according to the intervention logic and based on the monitored readings, if an intervention exit condition is present in the target person; and
if an intervention exit condition is present, dynamically reconfiguring the ventilator system to provide the base mechanical ventilation to the target person according to the base configuration.

3. The method of claim 1, wherein the base configuration and the intervention configuration each comprise one or more predetermined operational parameters and/or operating modes selected from the group consisting of: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T); Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations thereof.

4. The method of claim 2, wherein the base configuration and the intervention configuration each comprise one or more predetermined operational parameters and/or operating modes selected from the group consisting of: Continuous Positive Airway Pressure (CPAP), Bi-Level Positive Airway Pressure (BiPAP), Pressure control (PC), Volume-Limited Assist Control (AC), Synchronized intermittent Mandatory Ventilation (SIMV), Pressure Support Ventilation (PSV), Continuous Mandatory Ventilation (CMV), High Flow Nasal Therapy (HFNT), High Flow Oxygen Therapy (HFOT), or Spontaneous/Timed mode (S/T); Positive end expiratory pressure (PEEP), Pressure Support (PS), Respiratory rate (RR), Tidal volume (VT), Inspiratory airflow (V′), FiO2, Inspiratory positive applied pressure (IPAP), Peak inspiratory pressure (PIP), Inspiratory time, Inspiratory-to-expiratory ratio, Time of pause, Trigger sensitivity, Expiratory trigger sensitivity, Transpulmonary driving pressure (ΔP) and combinations thereof.

5. The method of claim 1 wherein the biometric sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

6. The method of claim 2 wherein the biometric sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

7. The method of claim 3 wherein the biometric sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

8. The method of claim 4 wherein the biometric sensor readings comprise readings selected from the group consisting of: heart rate, respiratory rate, blood pressure, Oxygen Saturation (SpO₂) End-Tidal Carbon Dioxide (ETCO₂), Minute Ventilation (V′), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

9. The method of claim 1 wherein the intervention logic comprises at lease one selected operational parameter and an entrance threshold for the selected operational parameter.

10. The method of claim 2 wherein the intervention logic comprises at lease one selected operational parameter, an entrance threshold for the selected operational parameter, and an exit threshold for the operational parameter.

11. The method of claim 3 wherein the intervention logic comprises at lease one selected operational parameter and an entrance threshold for the selected operational parameter.

12. The method of claim 4 wherein the intervention logic comprises at lease one selected operational parameter, an entrance threshold for the selected operation parameter, and an exit threshold for the operational parameter.

13. A dynamically configurable ventilator system for providing mechanical ventilation to a target person according to a prescription, configured to operate in accordance with the method of claim 1.

14. A dynamically configurable ventilator system for providing mechanical ventilation to a target person according to a prescription, configured to operate in accordance with the method of claim 2.

15. A dynamically configurable ventilator system for providing mechanical ventilation to a target person according to a prescription, configured to operate in accordance with the method of claim 4.
